(19) United States
(12) Patent Application Publication
(10) Pub. No.: US 2017/0188837 A1
(43) Pub. Date: Jul. 6, 2017

(54) OBJECT INFORMATION ACQUIRING APPARATUS, PHANTOM AND METHOD FOR MANUFACTURING SAME

(71) Applicant: CANON KABUSHIKI KAISHA, Tokyo (JP)

(72) Inventors: Yohei Hashizume, Tokyo (JP); Takahiro Suita, Kawasaki-shi (JP); Yoshie Ito, Sakura-shi (JP); Yasufumi Asao, Atsugi-shi (JP)

(73) Assignee: CANON KABUSHIKI KAISHA, Tokyo (JP)

(21) Appl. No.: 15/314,568

(22) PCT Filed: Jun. 10, 2015
(86) PCT No.: PCT/JP2015/067376

§ 371 Date: Nov. 29, 2016

(30) Foreign Application Priority Data
Jun. 17, 2014 (JP) 2014-124376
Jun. 17, 2014 (JP) 2014-124377
Jun. 17, 2014 (JP) 2014-124378
Jun. 17, 2014 (JP) 2014-124379
Jun. 17, 2014 (JP) 2014-124380
Jun. 17, 2014 (JP) 2014-124381

(51) Int. Cl. A61B 5/00; G01N 29/06; G01N 29/024; G01N 29/12; G01N 29/036; G01N 29/04; G01N 29/07; G01N 29/11; A61B 8/00; G01N 29/032

(52) U.S. Cl. CPC A61B 5/0095; A61B 8/587; A61B 5/0091; A61B 5/7203; G01N 29/0654; G01N 29/024; G01N 29/032; G01N 29/036; G01N 29/04; G01N 29/07; G01N 29/11; G01N 29/12; A61B 8/463; A61B 2560/0223

(57) ABSTRACT

Provided is an object information acquiring apparatus which uses a phantom having a base material, an absorber arranged inside the base material, and an identification tag, the apparatus including: a probe which receives an acoustic wave and outputs an electrical signal; a unit which reads in information from the identification tag; a unit which generates calibration information using an electrical signal based on an acoustic wave propagated from the phantom, and the read information; and a unit which acquires object information using the electrical signal based on the acoustic wave propagated from the phantom, wherein the identification tag stores information relating to the phantom.

### TECHNICAL FIELD

The present invention relates to an object information acquiring apparatus, a phantom and a method for manufacturing same.

### BACKGROUND ART

In the medical field, research has been advancing into object information acquiring apparatuses which create images of information relating to the inside of an object, such as a living body, by irradiating light, such as laser light, onto the object and receiving ultrasound waves generated from inside the object as a result of this light. When pulse laser light, or the like, is irradiated onto on object as irradiation light, an acoustic wave is generated when the irradiation light is absorbed by the tissue inside the object. By receiving and analyzing this acoustic wave, information relating to the optical characteristics of the inside of the object can be converted into an image. This phenomenon is called a photoacoustic effect, and the generated acoustic wave is called a photoacoustic wave. The technique using photoacoustic waves is called “photoacoustic tomography” (PAT).

When the quality of the acquired image is poor in an object information acquiring apparatus of this kind, then accurate diagnosis is not possible. Therefore, it is necessary for an object information acquiring apparatus to receive acoustic waves and to acquire an object image, with sufficient accuracy for the purpose of diagnosis. Consequently, it is necessary to carry out measurement at an accuracy corresponding to the specifications of the apparatus.

Furthermore, if the object is a living body, then since the object information acquiring apparatus irradiates laser light onto the living body, safety must be upheld. Therefore, it is necessary to maintain the operational state of the apparatus upon shipment, even when a certain time period has elapsed after the start of use.

In order to satisfy these requirements, measurement of a phantom for the object information acquiring apparatus is carried out by an operator or service staff, and calibration is carried out on the basis of the acquired image. The apparatus does not have to be calibrated each time photoacoustic measurement is carried out, but is desirably calibrated upon instalment of the apparatus, or each time the apparatus has been used a prescribed number of times, or each time a prescribed time period has elapsed.

Patent Document 1 discloses a QC (Quality Control) phantom for inspection of a radiation image reading apparatus, which includes a unit for holding identification information that identifies the type of QC phantom. The objective of this is to save the work of inputting the data computation parameters and/or data computation method, which differ for each type of QC phantom.

A phantom for an object information acquiring apparatus may also be provided with an identification information holding unit. Consequently, phantom identification information and initial information upon shipment is held in the phantom, and can be read by the object information acquiring apparatus.

### CITATION LIST

### Patent Literature

PTL1: Japanese Patent Application Publication No. 2004-223138

### SUMMARY OF INVENTION

### Technical Problem

The QC phantom disclosed in Patent Document 1 has problems such as the following with regard to the quality of the acquired phantom image, when acquiring an object image.

One problem is that if laser light is irradiated onto the identification information holding unit, while irradiating laser light onto the phantom, then a photoacoustic wave is also generated by the holding unit which has absorbed the laser light. The photoacoustic wave thus generated may become an artifact when converted into an image, thereby degrading the quality of the phantom image used in calibration.

A further problem is that positional information for tangible objects other than the object for evaluation, which are present in the phantom upon shipment of the phantom, are not considered in the identification information of the QC phantom disclosed in Patent Document 1.

Essentially, when a plurality of phantoms are manufactured under the same conditions, it is desirable for the initial state of the interior of the phantom base material to be uniform. However, in actual practice, as a result of thorough research by the present inventors, it was found that there are tangible objects (air bubbles, foreign matter, etc.) in the phantoms, when the phantoms are manufactured. Artifacts may be generated by these tangible objects, and there is a risk that the prescribed calibration may not be possible.

A further problem is that the initial shape information of the phantom upon shipment is not considered in the identification information of the QC phantom disclosed in Patent Document 1.

Essentially, when a plurality of phantoms are manufactured under the same conditions, it is desirable for the initial state, such as the shape of the phantom base material, to be uniform for the same model number. For example, if there is an abnormality (defect, omission, unevenness, etc.) in the surface profile of the phantom, then the circumstances relating to the incidence and diffusion of the light inside the phantom will not be as expected, thus affecting the intensity of the photoacoustic wave generated from the absorber.

However, in actual practice, fluctuations occur between manufacturing lots, due to causes such as slight differences in the state of the mold or the raw material used to manufacture the phantom, and this leads to error in the initial shape information. Furthermore, inside a single phantom, there may be a distribution in the state of surface unevenness, depending on the location, and there may be error in the initial shape information. Errors of this kind give rise to deterioration in the quality of the phantom image, such as the appearance of artifacts, or the like, and lead to decline in the accuracy of calibration.

A further problem is that, when the acoustic property value or optical property value of at least a portion of the base material of the phantom is different to that of other portions of the phantom, then this affects the state of transmission and scattering of the acoustic wave, and the light intensity, etc.

A further problem is that of increased costs when an IC tag, or the like, is used as an identification tag. In other words, a dedicated reading apparatus is required for reading the tag information, and the cost of the apparatus increases. In this respect, there is a method for forming an identification tag using lead, which has reflecting properties in a radiation image, in a radiation apparatus. However, a method wherein a material which is reflected in the measurement image in this way is embedded into the phantom has a problem in that the amount of information that can be transmitted thereby is small. In other words, when an identification tag is made using a material which is reflected in the measurement image, then the density cannot be raised above the resolution of the apparatus.

A further problem is that, since the photoacoustic measurement apparatus has a feature of irradiating measurement light onto the object and receiving an acoustic wave generated inside the object, then if measurement light is irradiated onto the identification tag, an acoustic wave is generated from inside the identification tag. Since this acoustic wave includes information that is unrelated to the phantom, then there is a risk that this information will appear as an artifact in the image when processed directly, thus impeding calibration.

The present invention was devised on the basis of recognizing problems of this kind. The object of the present invention is to improve the accuracy of calibration by reducing the effects of artifacts in an image acquired using a phantom for calibration.

### Solution to Problem

The present invention provides an object information acquiring apparatus which uses a phantom having a base material, an absorber arranged inside the base material, and an identification tag, the apparatus comprising:

a probe which receives an acoustic wave and outputs an electrical signal;

a reader which reads in information from the identification tag;

a generator which generates information for performing calibration, by using an electrical signal output by the probe as a result of receiving an acoustic wave propagated from the phantom, and information read out from the identification tag by the reader; and

a processor which acquires property information of the inside of an object by using the electrical signal output by the probe as a result of receiving the acoustic wave propagated from the phantom,

wherein the identification tag stores information relating to the phantom, the information being used by the processor when acquiring the property information.

The present invention also provides a phantom used for calibration of an object information acquiring apparatus which acquires property information of the inside of an object on the basis of an acoustic wave propagated from the object, the phantom comprising:

a base material;

an absorber arranged inside the base material; and

an identification tag which stores information relating to the phantom, the information being used by the object information acquiring apparatus when acquiring the property information.

The present invention also provides a method for manufacturing a phantom used for calibration of an object information acquiring apparatus which acquires property information of the inside of an object on the basis of an acoustic wave propagated from the object, the method comprising steps of:

forming a base material;

arranging an absorber inside the base material;

arranging an identification tag in the base material; and

storing information relating to the phantom in the identification tag, the information being used by the object information acquiring apparatus when acquiring the property information.

### Advantageous Effects of Invention

According to the present invention, it is possible to improve the accuracy of calibration by reducing the effect of artifacts in an image acquired using a phantom for calibration.

### BRIEF DESCRIPTION OF DRAWINGS

FIGS. 1A and 1B are schematic drawings showing a configuration of a phantom for an object information acquiring apparatus;

FIG. 2 is a schematic drawing showing a configuration of an object information acquiring apparatus;

FIG. 3 is a schematic drawing showing a phantom image;

FIGS. 4A to 4E are schematic drawings showing the contents of signal processing and a phantom image;

FIGS. 5A to 5C are schematic drawings showing a configuration according to a third embodiment;

FIGS. 6A to 6D are schematic drawings showing a configuration according to a third embodiment;

FIG. 7 is a schematic drawing showing a configuration relating to a fourth embodiment;

FIG. 8 is a flow diagram showing a method for implementing calibration;

FIG. 9 is a schematic drawing showing a configuration relating to a fifth embodiment;

FIG. 10 is a schematic drawing showing a configuration relating to a sixth embodiment;

FIGS. 11A and 11B are schematic drawings showing a configuration relating to a sixth embodiment;

FIGS. 12A and 12B are schematic drawings showing a configuration relating to a seventh embodiment;

FIGS. 13A and 13B are schematic drawings showing a configuration relating to an eighth embodiment;

FIGS. 14A and 14B are schematic drawings showing a configuration relating to an eighth embodiment; and

FIGS. 15A and 15B are schematic drawings showing a configuration relating to a ninth embodiment.

### DESCRIPTION OF EMBODIMENTS

Below, preferred embodiments of the present invention are described with reference to the drawings. The dimensions, materials, shapes and relative positions, and the like, of the constituent parts described in these embodiments should be changed appropriately depending on the composition and various conditions of the apparatus to which the invention is applied, and it is not intended to limit the scope of the invention to the description of the embodiments given below.

The present invention relates to technology for detecting an acoustic wave propagated from an object, and generating and acquiring property information of the inside of the object. Therefore, the present invention maybe understood as an object information acquiring apparatus or control method for same, or an object information acquiring method or a signal processing method. The present invention may also be understood as a program which causes an information processing apparatus provided with a CPU and/or other hardware resources to execute these methods, or a storage medium on which this program is stored. The present invention may also be understood as a phantom for calibration, or a method of manufacturing same, or a calibration method using the phantom.

The object information acquiring apparatus according to the present invention includes apparatuses using photoacoustic tomography which irradiates light (electromagnetic wave) onto an object, and receives (detects) an acoustic wave generated and propagated at a specific position inside the object or on the surface of the object, due to a photoacoustic effect. An object information acquiring apparatus of this kind is called a photoacoustic imaging apparatus or photoacoustic image-forming apparatus, since property information for the interior of the object is obtained in the form of image data, or the like, on the basis of photoacoustic measurement. An object information acquiring apparatus of this kind may also be called a photoacoustic tomography apparatus.

The property information in the photoacoustic apparatus indicates the distribution of a generation source of an acoustic wave which is generated by the irradiation of light, the initial sound pressure distribution inside the object, or a light energy absorption density distribution or absorption coefficient distribution derived from the initial sound pressure distribution, and the density distribution of the material constituting tissue. More specifically, the property information indicates the density distribution of oxidized/reduced hemoglobin, or the blood component distribution, such as the oxygen saturation distribution, derived from the hemoglobin density distribution values, or the distribution of fat, collagen, water, or the like. Furthermore, the property information may be determined as distribution information for each position inside the object, rather than as numerical data. In other words, the object information may be distribution information, such as the absorption coefficient distribution or oxygen saturation distribution, or the like.

The acoustic wave referred to in the present invention is typically an ultrasound wave, and includes elastic waves called sound waves or acoustic waves. An acoustic wave generated by the photoacoustic effect is called a photoacoustic wave or optical ultra-sound wave. An electrical signal converted from an acoustic wave by a probe is called an acoustic signal.

The object in the present invention is assumed mainly to be a human breast. However, the object is not limited to this and measurement of another segment of a living body, or of non-living material, is also possible.

### Method for Implementing Calibration

Firstly, a procedure of an apparatus configuration using a phantom for calibration, which is common in the present invention, will be described. Examples of the calibration items are listed below.

(1) Quantity of light irradiated by light source, light irradiation angle on object and irradiation position
(2) Transparency and positional accuracy of object holding member (plate, cup, etc.)
(3) Detection sensitivity, position and angle of acoustic wave probe (or element contained in the probe)
(4) Speed and positional accuracy of scanner
(5) Processing capacity of signal processing unit (amplification rate, A/D conversion frequency, image reconstruction accuracy, etc.)

Apart from this, any other adjustable parameter which is related to the measurement capability of the apparatus may be an object of calibration. These parameters may be input by a user via a GUI or physical input apparatus, or may be controlled automatically by an information processing apparatus.

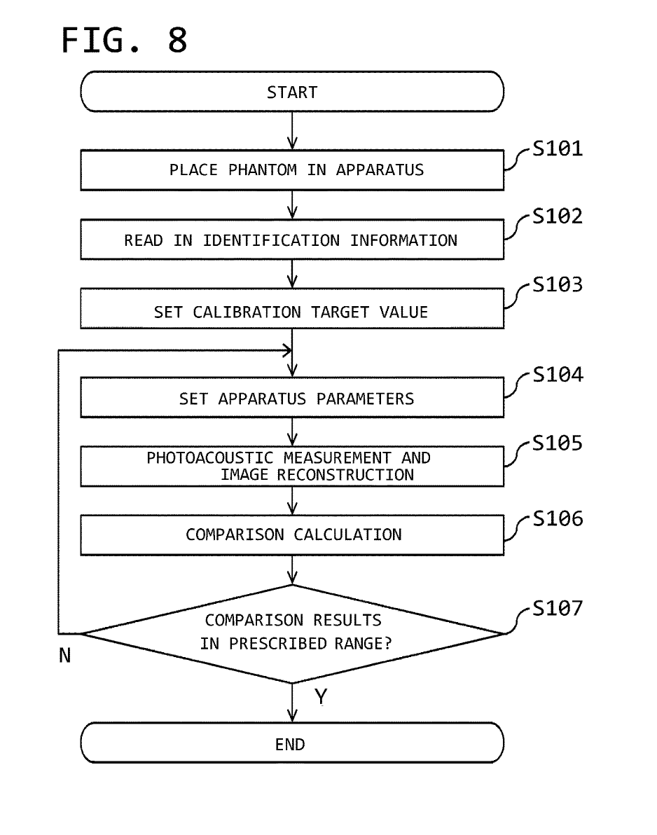

A general calibration procedure is now described with reference to the flowchart in FIG. 8.

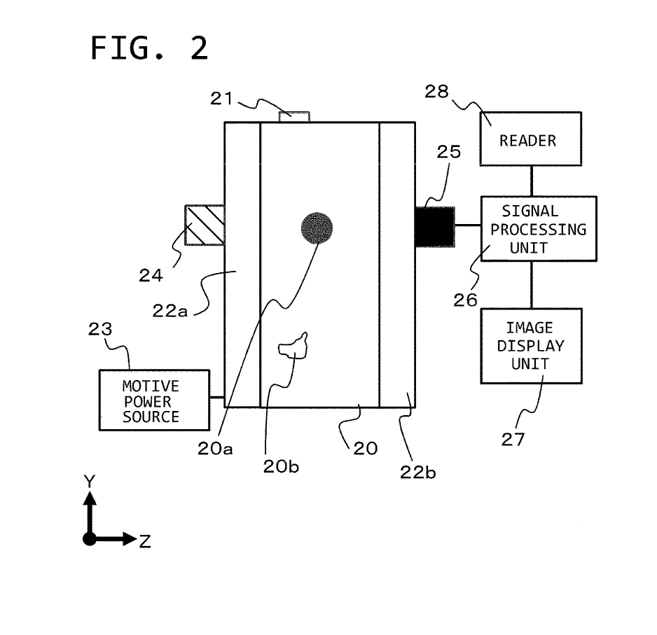

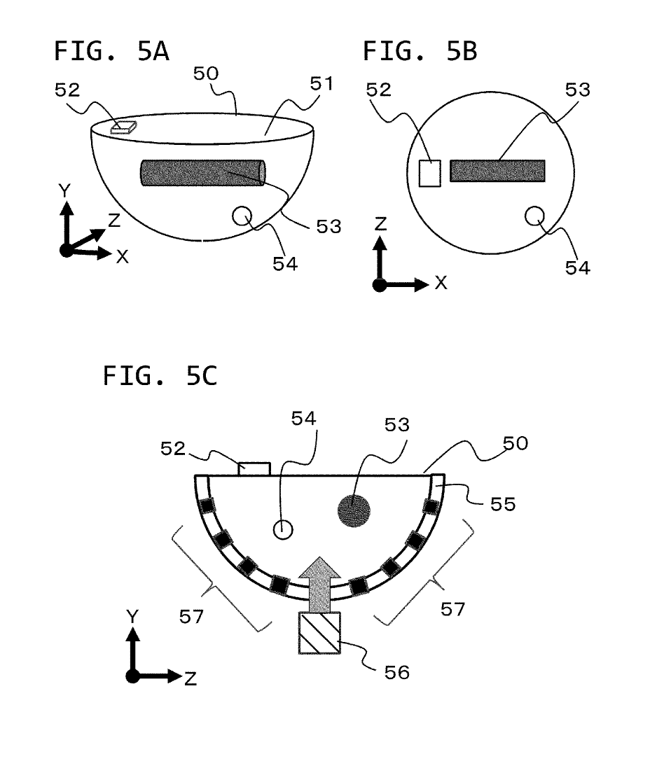

After starting a calibration process, in step S101, a phantom for calibration is placed in the apparatus. For example, in the case of the apparatus shown in FIG. 2, a phantom is placed between two plates, and the phantom is pressed and held by moving one of the plates. Furthermore, in the case of the phantom in FIGS. 5A to 5C, the phantom is held by a supporting body (for example, a cup-shaped holding member). Desirably, a positioning member is provided in the phantom to improve the accuracy of alignment.

In step S102, the information stored on an identification tag is read in by a reader of the apparatus. Here, at least the information required for the apparatus to acquire the characteristics of the phantom for calibration is read in. The information held may be phantom characteristics (size, material, spectral characteristics, absorber information, expected reconstructed image), which are held directly, or may be only phantom model number information, by which the apparatus separately acquires the phantom characteristics for that model number.

In step S103, the calibration target values are set. The calibration target values are, for example, data forming an ideal phantom image, which is stored in a memory in the signal processing unit or in the identification tag. Alternatively, the calibration target values may be generated by the signal processing unit on the basis of the identification information. This calibration target value data is data that allows an ideal phantom image after calibration to be displayed on the image display unit, supposing that a phantom image is displayed on the image display unit on the basis of the data. Alternatively, it is also possible to actually display an ideal phantom image on the image display unit, as a calibration target value. The calibration method used in this case is described below.

In step S104, various parameters are set for the object information acquiring apparatus in relation to the phantom. This is a task of setting concrete values for the calibration items indicated above. For example, in relation to the light source in (1), the amount of light, irradiation angle, irradiation position, and the like, to be applied in the next step S104, are determined respectively.

In step S105, photoacoustic measurement and image reconstruction are carried out and an image of the phantom is acquired. More specifically, light is irradiated onto the phantom from a light source, and a photoacoustic wave generated by a photoacoustic effect is received by a probe. Image reconstruction is then carried out by a known method, on the basis of the electrical signal originating from the photoacoustic wave.

In step S106, the calibration target value and the reconstructed image data are compared. This comparison involves comparing the respective brightness values, for example. In this comparison, well-known techniques such as creating a pixel value histogram, or edge detection and filtering, etc. can be used.

Thereupon, in step S107, it is determined whether or not the comparison result comes within a prescribed range of error. If the result is negative, then the apparatus parameters are corrected, and steps S104 to S106 are repeated until the comparison result comes within the prescribed range of error. On the other hand, if the result is affirmative, then prescribed termination processing, such as displaying the result, is carried out and calibration is then terminated. If the result does not come within the prescribed error after a set number of comparison calculations, then an error display is shown on the image display unit and the processing is terminated.

When the calibration target values form an ideal phantom image that is displayed on the image display unit, then a reconstructed image is also displayed on the image display unit, and calibration is carried out by visually comparing the ideal phantom image and the reconstructed image that is displayed. In this case, photoacoustic measurement is carried out after a human operator has manually input the apparatus parameters, and furthermore the operator makes a visual comparison of the actual phantom image and the ideal image, and adjusts the parameters accordingly. The image display unit corresponds to a display of the present invention.

In addition to this calibration flow, in the respective embodiments of the present invention, an identification tag of the phantom for calibration also stores information about tangible objects contained in the phantom (information such as the position, size, shape, optical properties, acoustic properties, and the like). By the apparatus reading in this information, processing for improving accuracy as indicated in the embodiments described below can be achieved. The tangible objects are objects other than the absorber, which is the object for imaging in the phantom, and may be air bubbles, foreign objects, conglomerations, or the like.

The flow described above is applied to the tag which stores this tangible object information. Calibration can be carried out basically by similar processing to that described above, even if the information stored in the tag includes different contents. The details of this are described in the various embodiments.

### First Embodiment

A phantom for an object information acquiring apparatus relating to a first embodiment is provided with an identification tag. The identification tag has positional information for tangible objects other than the object being assessed, which are present in the phantom. Here, the tangible objects are foreign objects, such as air bubbles, conglomerations of scattered material, shards of stirring blade, etc. If a tangible object which is a foreign object is present, then an unwanted photoacoustic wave is generated when light is irradiated, and therefore noise enters into the phantom image and the accuracy of calibration declines.

<Configuration of Phantom for Object Information Acquiring Apparatus>

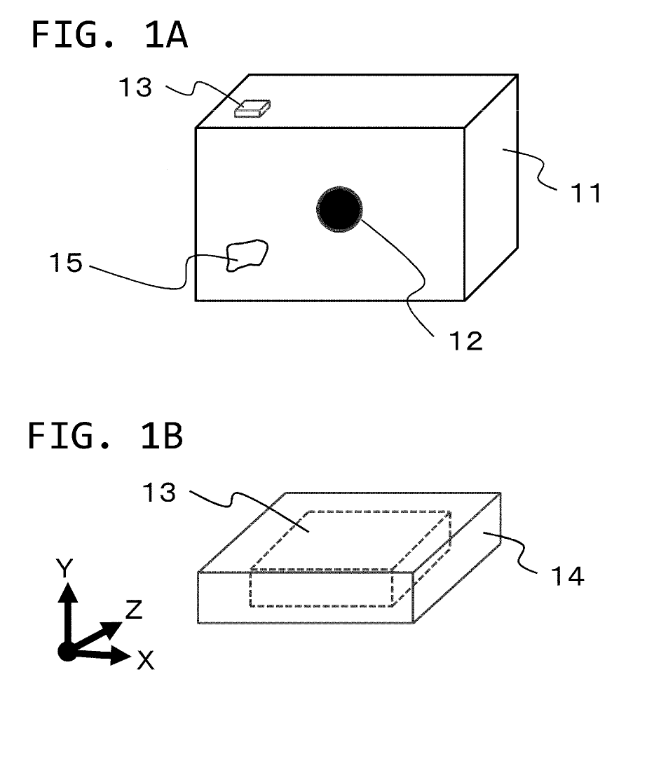

The configuration of the phantom for an object information acquiring apparatus relating to the first embodiment is now described with reference to FIGS. 1A and 1B. FIG. 1A is one example of a schematic drawing of a phantom, and FIG. 1B is one example of a schematic drawing of an identification tag. The phantom in FIG. 1A has a cuboid shape, and is formed to dimensions of 15 cm in the X direction, 10 cm in the Y direction and 5 cm in the Z direction. Below, a phantom of this shape is called a cuboid phantom.

Reference numeral 11 is a phantom base material. The material of the phantom base material is desirably a material that simulates the acoustic properties and optical properties of the object (for example, living tissue). For instance, the material may be polyol, or polyol in which a compound having light scattering or light absorptive properties is dispersed. The polyol used in the present invention is desirably a polyether polyol, from the viewpoint of compatibility with the acoustic properties of living tissue. Furthermore, a compound having light scattering properties is used in order to make a reduced scattering coefficient, which is one of the optical properties, approach that of living tissue. The particles of the compound are subjected to surface modification, or the like, in order to raise the uniformity of dispersion in the polyol. Desirably, when titanium oxide is used as a compound having light scattering properties, surface treatment is carried out using aluminum oxide and hexamethyl disilazane.

Reference numeral 12 is an absorber, and is desirably a member that simulates the acoustic properties and optical properties of a tumor. The material of the absorber 12 may be, in addition to the material of the phantom base material 11, a material in which pigment is dispersed as a compound having light absorptive properties. The pigment is used in order to approach the absorption coefficient of living tissue, which is one optical property, and it is suitable to employ carbon black, or the like, as the pigment.

Therefore, essentially, the most characteristic portion of the phantom image is the portion originating from the photoacoustic wave generated from the absorber 12. The brightness of the phantom image can be adjusted in accordance with the differential between the absorption coefficients of the phantom base material 11 and the absorber 12. When a foreign object (tangible object) or the identification tag is converted into an image, then characteristic features occur in different portions to an ideal image, and abnormalities in contrast occur.

Reference numeral 13 is an identification tag, which uses a well-known storage medium, such as a wireless IC tag or IC memory, a two-dimensional bar code, QR code, or the like. Positional information for tangible objects (in the present embodiment, conglomerations of titanium oxide) other than the assessment object, which are present in the phantom, is stored in the identification tag of the present invention. Furthermore, as described above, basically, various information required in order to create an ideal image for calibration is included in the identification tag. The positional information for the tangible objects is two-dimensional information represented by a XY coordinates system, or three-dimensional information represented by an XYZ coordinates system. The units of the respective coordinate values are units representing distance (cm or mm, etc.) or voxel numbers. The coordinate values may be single values, or may be set within a range.

In FIG. 1A, the identification tag 13 is disposed on the upper surface of the phantom in the Y direction, but the positioning of the tag is not limited to this. When a reflector 14, which is described below, is not provided, the surface of the identification tag 13 is desirably made of a material having high reflectivity in respect of the wavelength of the light irradiated by the object information acquiring apparatus, for example, a metal such as Ag, Cu or Al, or a dielectric multilayer film. When wavelengths in the near-infrared region are used for the irradiation light, the materials described above have a reflectivity of 80% or more.

Desirably, the information on the identification tag 13 is read in by a non-contact method. Therefore, a wireless IC tag may be used as the identification tag 13. The frequency band used is desirably in the range of several MHz to several GHz and most desirably, a range of 860 MHz to 960 MHz.

Reference numeral 14 is a reflector, which is disposed so as to cover the entire surface of the identification tag 13. The material used for the reflector is one having high reflectivity with respect to the wavelength of the light irradiated from the object information acquiring apparatus. Furthermore, the reflector may be coated directly onto the identification tag 13, or may be coated thereon after forming a protective layer on the surface of the identification tag 13.

Reference numeral 15 is a conglomeration of titanium oxide in the phantom base material, which occurs due to stirring non-uniformities during manufacture. This is a tangible object (foreign object) in the present embodiment.

<Method for Manufacturing Phantom for Calibration>

Next, a method for manufacturing a phantom for calibration according to the present embodiment will be described. The phantom base material 11 is polyol. This polyol is introduced into a beaker, and a compound having light scattering properties or light absorptive properties is dispersed in the beaker, and stirred, whereupon vacuum degassing is carried out. As the compound having light scattering properties, titanium oxide (average particle diameter 0.21 μm) which has been subjected to a surface treatment using aluminum oxide and hexamethyl disilazane is dispersed. During this dispersion, a conglomeration of the titanium oxide occurs in the phantom base material and becomes a tangible object (foreign object).

The absorber 12 is obtained by further dispersing a compound having light absorptive properties in the abovementioned material of the phantom base material 11. A mixture of carbon black pigment and polyether dispersing agent having affinity with the pigment is used as this compound. The polyol used is a polyether-polyol copolymer (number average molecular weight 6000) having a mol ratio of 1:1 between ethylene oxide and propylene oxide.

The size of the absorber 12 is a sphere of 5 mm diameter, and the position of the absorber 12 within the phantom base material is set to 7.5 cm in the X direction, 5 cm in the Y direction and 2.5 cm in the Z direction, by using transparent nylon wire (diameter 0.13 mm) which does not generate a photoacoustic wave when light is irradiated thereon.

Thereupon, hexamethylene diisocyanate (HDI), which is a curing agent, is added to the polyol and stirred, vacuum degassing is carried out, and the material is poured into a prescribed mold and thermally cured.

The physical values of a configured phantom, which are well-known from documents, etc. are indicated below. The documented acoustic properties of the phantom base material 11, at 37° C., are a speed of sound of 1379 m/s, an acoustic impedance of 1.42 rayl, and an acoustic attenuation of 0.52 dB/cm/MHz. The documented optical properties are an absorption coefficient of 0.004/mm and a reduced scattering coefficient of 0.93/mm, at a wavelength of 797 nm. Furthermore, the documented acoustic properties of the absorber 12, at 37° C., are a speed of sound of 1508 m/s, an acoustic impedance of 1.63 rayl, and an acoustic attenuation of 1.08 dB/cm/MHz. The documented optical properties are an absorption coefficient of 0.098/mm and a reduced scattering coefficient of 0.92/mm, at a wavelength of 797 nm.

A wireless IC tag is used for the identification tag 13, and stores the date of manufacture of the phantom, the site of manufacture, the production lot number, and positional information for tangible objects other than the assessment object in the phantom. After a silicon nitride film has been formed to 500 nm as a protective layer on the surface of the identification tag, the entire surface of the tag is covered using Ag paste as a reflector 14. Thereupon, the tag is fixed by adhesive to the upper surface of the phantom in the Y direction.

<Configuration of Object Information Acquiring Apparatus>

The configuration of the object information acquiring apparatus relating to the first embodiment is now described with reference to FIG. 2. FIG. 2 is a schematic drawing showing an example of the configuration when using a cuboid phantom as an object of measurement.

Reference numeral 20 is a cuboid phantom, and 21 is a identification tag provided on the cuboid phantom. Reference numeral 20a is an absorber disposed inside the phantom 20. Reference numeral 20b is a conglomeration of titanium oxide.

Reference numerals 22a and 22b are two holding plates. Reference numeral 23 is a motive power source. By moving at least one of the two holding plates by motive power generated by the motive power source 23, the cuboid phantom 20 is pressed and held.

Reference numeral 24 is a light source which generates light that is irradiated onto the cuboid phantom 20, and light irradiated from this light source 24 (pulse light) is irradiated onto the cuboid phantom 20 through the holding plate 22a.

Reference numeral 25 is an acoustic wave probe which receives a photoacoustic wave generated from the cuboid phantom 20, via the holding plate 22b. Reference numeral 26 is a signal processing unit which generates image data by processing the signal received by the acoustic probe 26, and reference numeral 27 is an image display unit which displays image data generated by the signal processing unit 26. Reference numeral 28 is a reader which reads in information in the identification tag 21. The information read in by the reader 28 is displayed on the image display unit 27.

The constituent elements of the apparatus relating to the present embodiment are described below.

<<Holding Plates 22>>

The holding plates 22a and 22b are two plate-shaped holding members for holding an object, and the object can be pressed and held by moving at least one of the plates by a motive power generated by the motive power source 23.

The material of the holding plate 22a which is positioned on the side of the light source 24 is desirably a material having high transmissive properties and low attenuation properties with respect to the light irradiated from the light source 24. For example, the material is glass, polymethyl pentene, polycarbonate, acrylic, or the like.

Furthermore, the holding plate 22b positioned on the side of the acoustic wave probe 25 must transmit the acoustic wave generated inside the cuboid phantom 20, and therefore is desirably made of a material whereby the acoustic wave is not liable to be reflected at the interface with the cuboid phantom 20. More specifically, a desirable material is one having a small differential in acoustic impedance with respect to the object, which is the object of measurement of the present apparatus. For example, the material may be a resin material, such as polymethyl pentene.

The holding plate 22 desirably has a thickness which maintains a certain strength, in such a manner that the plate is not deformed by the holding pressure, and typically, a thickness of approximately 10 mm is desirable. Furthermore, the holding plate 22 may be of any size, provided that the plate is capable of holding the object, but is desirably of a similar size to the object.

The motive power source 23 is desirably configured by a motor, and it is possible to use a DC motor, AC motor, stepping motor, or the like.

<<Light Source 24>>

The light source 24 is an apparatus which generates pulse light that is irradiated onto the object. The light source is desirably a laser light source in order to obtain a large output, but it is also possible to use a light-emitting diode or a flash lamp, or the like, instead of the laser. When a laser is used as a light source, it is possible to use various lasers, such as a solid laser, gas laser, dye laser, semiconductor laser, or the like. Furthermore, in order to generate a photoacoustic wave effectively, the light must be irradiated for a sufficiently short time in accordance with the thermal properties of the object. When light is irradiated onto the object, the pulse width of the pulse light generated from the light source is desirably 10 to 50 nanoseconds, approximately. Furthermore, the wavelength of the pulse light is desirably a wavelength at which light is propagated inside the object. More specifically, when the object is a living body, the wavelength is desirably no less than 650 nm and no more than 1100 nm. Moreover, the wavelength of the pulse light is desirably one having a high absorption coefficient in relation to the measurement object.

<<Acoustic Wave Probe 25>>

The acoustic wave probe 25 receives a photoacoustic wave generated inside the object and the cuboid phantom 20 as a result of light irradiated thereon, and converts the photoacoustic wave into an analogue electric signal. The acoustic wave probe 25 receives an acoustic wave generated inside the object and inside the cuboid phantom 20.

The acoustic wave probe 25 may be called simply “probe” or “transducer”. The acoustic wave probe 25 may be constituted by a single acoustic detector or by a plurality of acoustic detectors. Furthermore, the acoustic wave probe 25 may have a plurality of receiving elements arranged in a one-dimensional or two-dimensional configuration. When using a multi-dimensional element arrangement, the acoustic wave can be received simultaneously in a plurality of locations, and therefore the measurement time can be shortened and furthermore the effects of vibration of the object, and the like, can be reduced.

Moreover, when the light source 24 described above can be made to perform a scanning action by a scanner, desirably, the acoustic wave probe 25 is also made to perform a scanning, simultaneously with the light source 24, by the scanner.

<<Signal Processing Unit 26>>

The signal processing unit 26 amplifies the analog electrical signal obtained by the acoustic wave probe 25 and converts the electrical signal into a digital signal. The signal processing unit 26 is configured typically by an amplifier, an A/D converter, a FPGA (Field Programmable Gate Array) chip, and the like. When there is a plurality of detection signals obtained from the probe, then it is desirable for the signal processing unit 26 to be capable of processing a plurality of signals simultaneously. In order to achieve simultaneous processing, a plurality of channels is required. An amplifier may be incorporated inside the acoustic wave probe 25.

Furthermore, the signal processing unit 26 generates (reconstructs) image data by processing the acquired digital signal. For the image reconstruction method which is implemented by the signal processing unit 26, it is possible to adopt a Fourier transform method, a universal back projection method, a filtered back projection method, a progressive reconstruction method, or the like, but any kind of image reconstruction method may be used. An information processing apparatus which operates according to a program, such as a computer or workstation, etc., can be used as the signal processing unit 26. The functions achieved by the signal processing unit correspond to the functions performed by the generator and the processor of the present invention.

<<Image Display Unit 27>>

The image display unit 27 is a unit for displaying the reconstructed image generated by the signal processing unit 26 and the positional information for a tangible object in the phantom which has been read in by the reader 28. The image display unit 27 may use a liquid crystal display, a plasma display, an organic EL display, a FED, or the like.

<<Reader 28>>

The reader 28 is a unit for reading in information on the identification tag 21. A commonly known reader is selected, as appropriate, depending on the type of identification tag 21. For example, if a wireless IC tag is used as the identification tag 21, then it is suitable to select a wireless IC tag reader as the reader 28.

<Measurement Method>

Next, a measurement method according to the present embodiment will be described. Firstly, the object (during calibration, the phantom for calibration, and during diagnosis, a living breast) is pressed and held by the holding plates 22. Thereupon, light pulses emitted from the light source 24 are irradiated onto the object. When a portion of the energy of the light propagated inside the object is absorbed by a light absorber, such as blood, then a photoacoustic wave is generated from the light absorber, due to thermal expansion. If cancer is present inside the object, then in the new blood vessels of the cancer, the light is absorbed in a specific fashion, similarly to the blood in the remaining normal parts, and a photoacoustic wave is generated. The photoacoustic wave generated inside the object is received by the acoustic wave probe 25.

The electric signal output by the acoustic wave probe 25 is analyzed by the signal processing unit 26. The analysis results are converted into image data representing property information of the inside of the object, and are output via the image display unit 27.

If the object is a phantom, then information about the absorber in the phantom is converted into an image by a similar measurement method, and is displayed on the image display unit 27.

<Measurement Results>

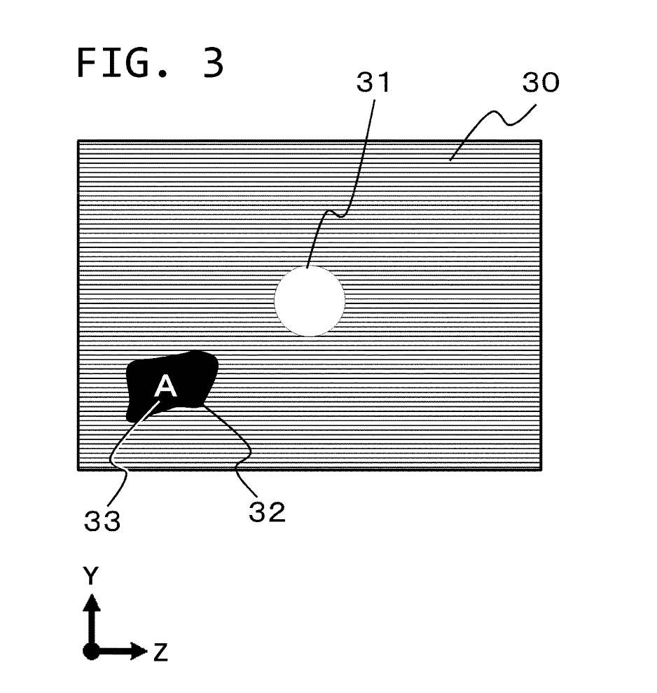

The measurement results are now described with reference to FIG. 3. FIG. 3 shows a phantom image which has been measured by the configuration in FIG. 2. Reference numeral 30 is a phantom image which is displayed on the image display unit 27. Reference numeral 31 is a signal generated from the absorber, which is displayed as brightness information corresponding to the absorption coefficient, on a dark grey background. A conglomeration of titanium oxide inside the phantom base material is displayed by a blacked-out portion such as region 32, on the phantom image. This is because the irradiation light is scattered strongly and therefore a photoacoustic wave is not generated.

On the basis of the coordinates of the tangible object in the phantom which is read out from the identification tag 21 by the reader 28, the image display unit 27 displays a text character “A”, which indicates artifact, at a corresponding position on the phantom image, as indicated by reference numeral 33. Therefore, the user is able to recognize that the blacked-out region 32 is an artifact.

Apart from this, the method for reporting the artifact position to the user may also involve placing coordinate axes on the phantom image and indicating the coordinates of the artifact in the display area. When the pointer is placed over the blacked-out region 32 which indicates an artifact, a display indicating that the region is an artifact may be provided. The region of the artifact may be surrounded by a dotted line, or the like. Furthermore, the details of the artifact (in this embodiment, a conglomeration of titanium oxide) maybe displayed alongside. Any display method may be adopted, provided that the method enables a user to recognize the position of the artifact.

Furthermore, even if the apparatus is carrying out automatic calibration, it is possible to achieve an accurate comparison by previously recognizing the position of the artifact.

The positional information of the artifact caused by a foreign object of this kind can be used for setting the parameters of the apparatus during calibration, for example. In other words, by using calibration target values and/or an ideal phantom image, which include an artifact, in S103 of the flow, the differential between the actual phantom measurement data (or phantom image) obtained using the set apparatus parameters, and the ideal values, becomes smaller. As a result of this, accurate parameter settings can be achieved. For instance, in the loop for correcting the apparatus parameters in the flow, the numerical values are corrected by taking account of the position of the artifact.

Alternatively, by using a calculation which incorporates the positional information of a conglomeration, in the comparison made in S106, then the accuracy of the comparison is improved and precise parameter settings can be achieved. For instance, if model number information is stored on the identification tag, and the signal processing unit obtains information about the phantom by referring to the model number in catalogue information, then according to the present embodiment, information about a conglomeration that cannot be obtained from the catalogue information is acquired. As a result of this, it becomes possible to set accurate parameters for the amount of light, the sensitivity of the acoustic probe, and so on.

Moreover, since the identification tag 21 is configured as described above, then when the cuboid phantom 20 is measured, it is possible to acquire a phantom image which is not of degraded quality, by avoiding the effects of the artifact caused by the identification tag 21.

Consequently, according to the measurement method using the apparatus according to the present embodiment, it is possible to reduce artifacts which arise from an identification information holding unit, such as an identification tag. Furthermore, it is possible to display the positions of the artifacts which are caused by tangible objects that are unique to each phantom, and to make the signal processing unit recognize these positions. Therefore, the accuracy of apparatus calibration using the phantom is improved, and the reliability of the image obtained by actually measuring the living body is improved, and therefore better diagnosis can be achieved.

### Second Embodiment

<Configuration of Phantom>

The configuration of the phantom for an object information acquiring apparatus relating to the second embodiment is now described with reference to FIG. 2. There are two points of difference with respect to the phantom for an object information acquiring apparatus according to the first embodiment. The first point of difference is that reference numeral 20b is a foreign object which generates a photoacoustic signal. Here, the foreign object is a shard of a blade. The second point of difference is that the identification tag 21 stores the date of manufacture of the phantom, the site of manufacture, the production lot number, position information for shaped objects other than the assessment object in the phantom, and photoacoustic wave shape information for a signal generated from a foreign object.

<Configuration of Object Information Acquiring Apparatus>

The object information acquiring apparatus relating to the second embodiment is now described with reference to FIG. 2. A portion of the details of the processing by the signal processing unit differs from the first embodiment.

The signal processing unit 26 according to the present embodiment has a function for identifying and deleting the waveform of the photoacoustic wave generated from a tangible object in a phantom, on the basis of the information on the identification tag 21. More specifically, the signal processing unit 26 estimates a region in the photoacoustic signal which contains the waveform of the tangible object on the basis of the sound speed value of the phantom base material and the positional information for the tangible object. In the estimated region, the waveform portion of the tangible object is identified by pattern recognition based on the photoacoustic waveform information. The identified waveform portion of the tangible object is deleted by masking with the surrounding waveform.

<Measurement Results>

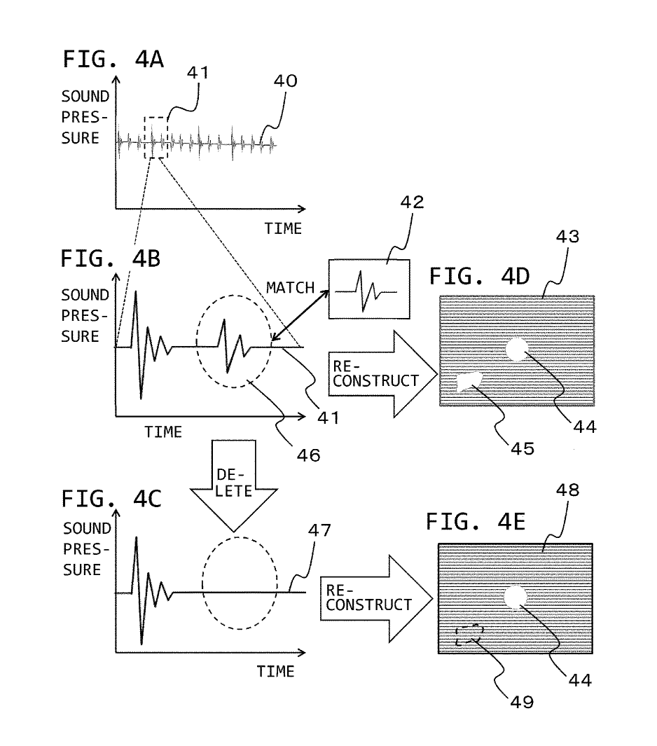

The measurement results are now described with reference to FIGS. 4A to 4E. FIGS. 4A to 4E show the process of the signal processing unit 26 deleting a waveform of a tangible object in a photoacoustic signal measured by the configuration in FIG. 2, using the information in the identification tag, and the display images when the waveform is deleted and when the waveform is not deleted.

In FIG. 4A, reference numeral 40 is a measured photoacoustic signal. The signal processing unit 26 estimates the region 41 within the signal that contains the photoacoustic wave generated from the tangible object in the phantom, on the basis of positional information for a blade shard recorded in the identification tag, and the sound speed value in the phantom base material 11. FIG. 4B shows an enlarged view of the region 41.

Next, the signal processing unit 26 identifies a waveform 46 matching a template 42 of the waveform shape of a foreign object, in the estimated region 41, and deletes the waveform by masking with the surrounding waveform. In this way, the artifact component is erased, as indicated by reference numeral 47 in FIG. 4C.

Reference numeral 43 in FIG. 4D is a phantom image which has been reconstructed from the region 41 by the signal processing unit 26 and which has not been subjected to the deletion described above. A signal 45 generated from the blade shard is displayed apart from the signal 44 generated from the absorber 20a, and prescribed calibration based on the phantom image is impeded.

Reference numeral 48 in FIG. 4E is a phantom image which has been reconstructed from the signal 47 after removal of the artifact component, and which has been subjected to the deletion described above. Only the signal 44 generated by the absorber 20a is displayed with contrast, and the signal 49 generated from the blade shard is masked by the surrounding signal and is not displayed.

By deleting the artifact generated from a tangible object that is unique to each phantom, as indicated in the method described above, the accuracy of the prescribed calibration used the phantom for calibration is improved. Furthermore, artifacts generated from the identification tag can also be reduced, similarly to the first embodiment.

The template 42 of the waveform shape of the foreign object has an effect in raising the accuracy, but is not essential. When the template 42 of the waveform shape is not stored in the identification tag, the region containing the photoacoustic wave generated from the tangible object in the phantom, which is estimated from the positional information and the speed of sound, is masked by the surrounding waveform and thereby deleted.

### Third Embodiment

A third embodiment of the present invention is described below. The main difference of the present embodiment with respect to the foregoing is the shape of the phantom for calibration.

<Configuration of Phantom for Object Information Acquiring Apparatus>

The configuration of the phantom for calibration will be described here with reference to FIG. 5A. Reference numeral 50 is a phantom, inside which there is a phantom base material 51 and an absorber 53, and an identification tag 52 is arranged on the upper surface of the phantom in the Z direction. Reference numeral 54 indicates a foreign object (here, an air bubble).

The phantom of the present embodiment has two points of difference with respect to the phantom of the second embodiment. The first point of difference is that the phantom has a hemispherical shape, and is manufactured to a diameter of 150 mm and a height of 100 mm. The second point of difference is that a cylindrical absorber is arranged inside the phantom. Below, a phantom of this shape is called a hemispherical phantom.

A phantom shape of this kind corresponds to the method for holding an object in the object information acquiring apparatus. More specifically, the apparatus of the present embodiment supports the breast by a cup-shaped or bowl-shaped holding member, rather than pressing and holding the breast by two plates.

Here, the phantom is a hemispherical body, but the actual shape thereof is not limited to this, and may be determined in accordance with the shape of the breast holding member or the expected size of the breast. For example, the phantom may have a spherical cap shape, which is shallower than a hemisphere. Furthermore, the phantom may also be shaped as a truncated portion of an elliptical body. Apart from this, the shape of the phantom can be determined in accordance with the shape of the breast and the holding member for same.

<Method for Manufacturing Phantom>

Below, a method for manufacturing a phantom for an object information acquiring apparatus relating to an embodiment of the present invention is described. The main differences with respect to the first embodiment are indicated below. FIG. 5A is an overall schematic drawing, FIG. 5B is a plan view perspective diagram, and FIG. 5C is a schematic cross-sectional diagram.

In FIG. 5C, reference numeral 55 is a spherical surface type holding section, which holds the whole spherical surface of the hemispherical phantom 50. A material having a strength capable of supporting the object and the hemispherical phantom 50 is selected as the spherical surface type holding section 55. In the present embodiment, acrylic having a diameter of 150 mm and a thickness of 10 mm is used as the material.

Reference numeral 56 is a light source which is arranged below the spherical surface type holding section 55, and irradiates light vertically upwards. Reference numeral 57 is an acoustic wave probe, in which elements are arranged in a hemispherical shape on the surface of the hemispherical surface type holding section 55 that contacts the object. In the acoustic wave probe 57, the diameter of the individual elements used is 2 mm, and these elements are arranged at 5 mm intervals apart on the surface of the spherical surface type holding section 55 that contacts the object. Reference numeral 53 is a cylindrical absorber having a diameter of 5 mm, which is manufactured from the same material. Reference numeral 54 is an air bubble which is mixed inside the material of the phantom by stirring during the manufacture of the phantom.

<Configuration of Object Information Acquiring Apparatus>

The configuration of the object information acquiring apparatus relating to a third embodiment is now described with reference to the differences with respect to FIG. 2. The apparatus of the present embodiment differs from FIG. 2, firstly, in respect of the object holding method and the shape of the phantom, as described above.

Moreover, the signal processing unit 26 according to the present embodiment has a function for specifying the image segment corresponding to the tangible object in a phantom in the phantom image, on the basis of the information in the identification tag 52, and for deleting this image segment by masking with the surrounding image.

<Measurement Results>

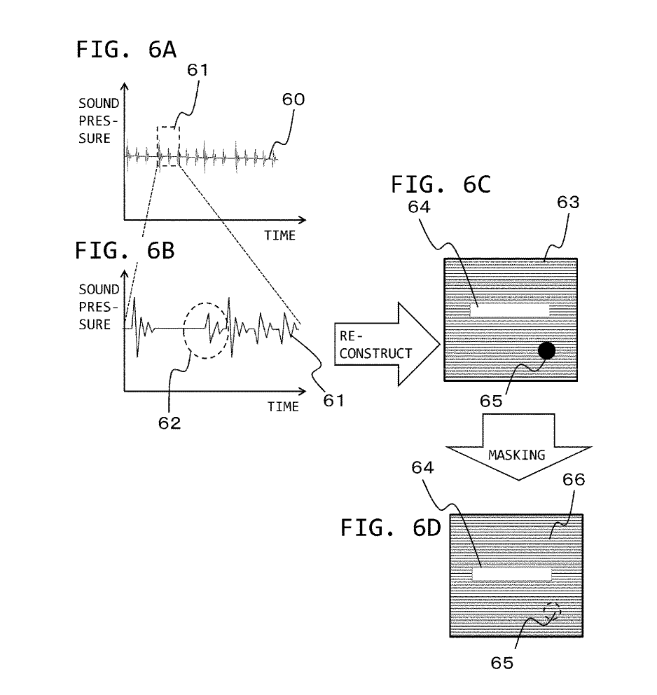

The measurement results are now described with reference to FIGS. 6A to 6C. FIGS. 6A to 6C show: the results obtained by measuring the phantom in FIGS. 5A to 5C; the process of deletion of an image segment caused by a tangible object, from a phantom image, which performed by the signal processing unit using information in the identification tag 52; and display images when deletion is performed and when deletion is not performed.

FIG. 6A is a graph including a photoacoustic wave signal 60 when the phantom in FIGS. 5A to 5C is measured. FIG. 6B shows an enlarged view of the region 61. The signal processing unit creates the reconstructed image 63 shown in FIG. 6C from this signal. In this image, an air bubble 54 is present as a blacked-out image region 65.

Furthermore, the signal processing unit estimates the region within the signal that contains the photoacoustic wave 62 generated from the tangible object in the phantom, on the basis of positional information for air bubbles recorded in the identification tag 52, and the sound speed value in the base material. Next, the signal processing unit calculates which region of the reconstructed image 63 the estimated region corresponds to, and deletes this region by masking with the image of the surrounding region. The image after deletion is as shown in FIG. 6D, and it can be seen that the artifact has been eliminated.

Reference numeral 66 in FIG. 6D is a phantom image after the signal processing unit has carried out the abovementioned deletion process on the reconstructed image 63. Only the signal 64 generated from the absorber 53 is displayed with contrast. On the other hand, the image region 65 which was displayed by the blacked-out region caused by the air bubble has been masked by the surrounding image.

Moreover, when the hemispherical phantom 50 is measured by the measurement method described above, it is possible to obtain a phantom image which his not affected by an artifact caused by the identification tag 52 and which does not have degraded image quality.

From the foregoing, according to the present embodiment, by reducing artifacts caused by the identification information holding unit, and deleting artifacts caused by tangible objects which are unique to each phantom, it is possible to carry out prescribed calibration on the basis of an image of the phantom.

### Fourth Embodiment

<Configuration of Phantom>

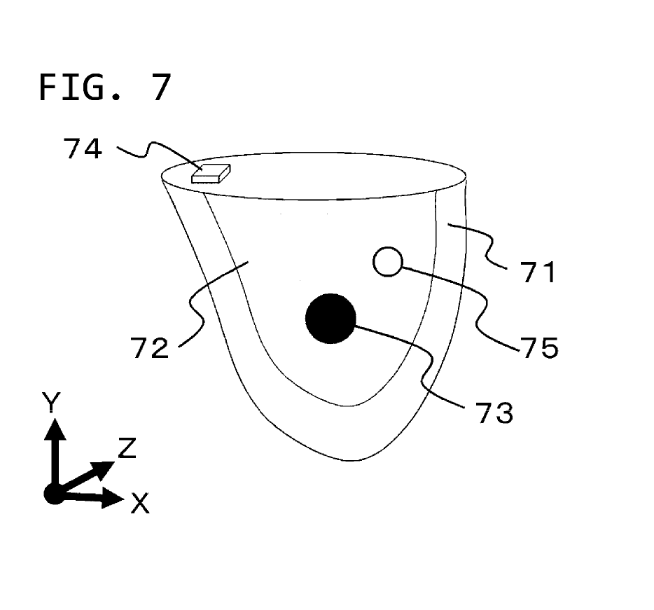

The configuration of the phantom for an object information acquiring apparatus relating to a fourth embodiment is now described with reference to FIG. 7. Reference numeral 71 is a simulated fat layer, reference numeral 72 is a simulated mammary layer, reference numeral 73 is an absorber, and reference numeral 74 is an identification tag. The identification tag 74 is disposed on the upper surface of the phantom in the Z direction. The difference with respect to the phantom for an objection information acquiring apparatus in the first embodiment is that the phantom is breast shaped, and the phantom base material has a layer structure comprising a plurality of materials. Below, a phantom of this shape is called a breast type phantom. Reference numeral 75 indicates a foreign object and here, is an air bubble.

<Method for Manufacturing Phantom>

Below, a method for manufacturing a phantom for an object information acquiring apparatus relating to an embodiment of the present invention is described. The simulated fat layer 71 is manufactured similarly to the phantom base material 11 of the first embodiment. More specifically, a convex-shaped mold is placed on a concave-shaped mold which is formed in a prescribed breast shape, the convex-shaped mold having an outer profile 10 mm smaller than the concave-shaped mold, and polyol without any added HDI is made to flow in between the concave-shaped and the convex-shaped molds and is thermally cured. Accordingly, a simulated fat layer 71 having a thickness of 10 mm is formed. Subsequently, a simulated mammary layer 72 is formed by causing polyol having added HDI to flow onto the concave-shaped mold in which the simulated fat layer 71 has been formed and curing the polyol.

The size of the absorber 73 is a 5 mm-diameter sphere, which is disposed inside the simulated mammary layer 72 using transparent nylon wire (diameter 0.13 mm) that does not generate a photoacoustic wave due to irradiation of light.

The physical values of the configured phantom are documented values. The acoustic properties of the simulated fat layer 71, at 37° C., are a speed of sound of 1379 m/s, an acoustic impedance of 1.42 rayl, and an acoustic attenuation of 0.52 dB/cm/MHz. The optical properties are an absorption coefficient of 0.004/mm and a reduced scattering coefficient of 0.93/mm, at a wavelength of 797 nm. The acoustic properties of the simulated mammary layer 72, at 37° C., are a speed of sound of 1496 m/s, an acoustic impedance of 1.52 rayl, and an acoustic attenuation of 0.84 dB/cm/MHz. The optical properties are an absorption coefficient of 0.011/mm and a reduced scattering coefficient of 1.02/mm, at a wavelength of 797 nm. Furthermore, the acoustic properties of the absorber, at 37° C., are a speed of sound of 1508 m/s, an acoustic impedance of 1.63 rayl, and an acoustic attenuation of 1.08 dB/cm/MHz. The optical properties are an absorption coefficient of 0.098/mm and a reduced scattering coefficient of 0.92/mm, at a wavelength of 797 nm.

<Measurement Results>

The flow during calibration and the method of using the identification tag information in the present embodiment are similar to the various embodiments described above. In the present embodiment, similarly to the various embodiments described above, the artifacts generated from the identification information holding unit can be reduced. Furthermore, by displaying the positions of artifacts generated from tangible objects which are unique to each phantom, and deleting the artifacts from the phantom image, it is possible to carry out the prescribed calibration accurately.

### Fifth Embodiment

The phantom for an object information acquiring apparatus relating to the fifth embodiment is provided with an identification tag having phantom shape information upon shipment (typically, surface profile information of the phantom). The method for specifying the profile of the phantom may be any method that enables the three-dimensional shape to be expressed. For example, there is a method in which the phantom is arranged in a prescribed virtual mesh, and the display position is specified by coordinates. Furthermore, the surface profile information may also be held as vector image data which can be subjected to information processing. Alternatively, the information may hold the differences with respect to a standard profile of a phantom of that model number.

If the surface profile is different to that expected, then the manner of incidence of the light into the object is also different to expectations. As a result of this, it becomes impossible to estimate accurately the quantity of light in a target voxel when determining the absorption coefficient from the initial sound pressure during image reconstruction, and therefore adverse effects in the image quality are inevitable. Moreover, when there are omissions, defects, or portions of non-uniform quality in the phantom, then an artifact occurs in the reconstructed image. When these phenomena occur in the phantom image during measurement of the phantom for calibration, the accuracy of calibration declines. Therefore, performing image reconstruction which takes account of the surface profile of the phantom, and outputting and displaying abnormalities in the surface profile of the phantom, on a display, are important in order to achieve accurate calibration.

In the present embodiment, in particular, phantom outer profile information at standard temperature (25° C.) absorber outer profile information, phantom surface unevenness information, and light distribution correction information, are stored in the identification tag as initial information upon shipment. Apart from this, any information regarded to be surface profile information may be stored in the IC tag. The standard temperature is a temperature corresponding to the temperature during manufacture of the phantom, for example. The acoustic properties and optical properties of the phantom vary with the temperature, and therefore accuracy can be improved by using the standard temperature as a condition in calibration. The information relating to the standard temperature is not necessarily applied in the present embodiment. The information held by the information tag may be any information, and if the temperature of the phantom affects the generated property information values, then the image quality can be improved by using the acquired standard temperature value.

Rather than indicating the position of an artifact obtained from an identification tag, on the screen, it is also possible to complement for abnormalities in the surface profile, when generating an image of a phantom. Accordingly, a user who is carrying out calibration is able to perform calibration based on the model number of the phantom, without being made aware of the differences in the surface profile. Image reconstruction is carried out on the basis of the phantom outer profile information, absorber outer profile information, phantom surface unevenness information, and light distribution correction information stored in the identification tag, and it can be confirmed that the measurement value from the phantom, and the calculated value, are matching in respect of the absorption coefficient of the absorber.

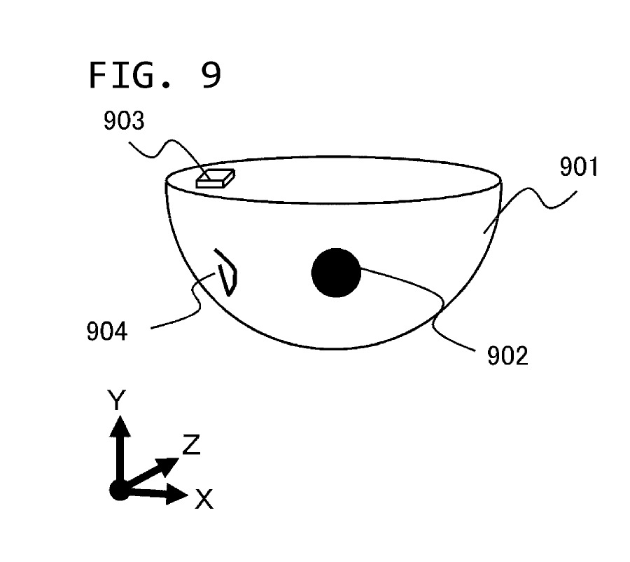

Provided inside the phantom for calibration in FIG. 9 are: a phantom base material 901 and an absorber 902; and an identification tag 903 is arranged on the upper surface of the phantom in the Z direction. Reference numeral 904 indicates a crack which has occurred in the phantom manufacturing process. When a crack caused by the manufacturing process occurs in the surface of the phantom, then the position of the crack in the phantom is read in as initial information, and the artifact arising from the crack position may be deleted.

Compared to the calibration flow in FIG. 8, the calibration flow of the present embodiment reflects the fact that the identification tag of the phantom for calibration stores various information relating to the outer profile of the phantom for calibration. For example, the outer profile itself may be stored as three-dimensional data, such as coordinates. Alternatively, the difference between the outer profile expected from a specific model number and the actual outer profile of the phantom may be stored. A difference of this kind may occur due to fine fluctuations in the conditions during manufacture of the phantom (for example, the accuracy of the mold, the quality of the raw material, the accuracy of manufacturing process control during solidification of the base material). Furthermore, omissions, defects, and fine unevenness may occur after manufacture of the phantom. The image accuracy is improved by the apparatus reading in this outer profile information.

### Sixth Embodiment

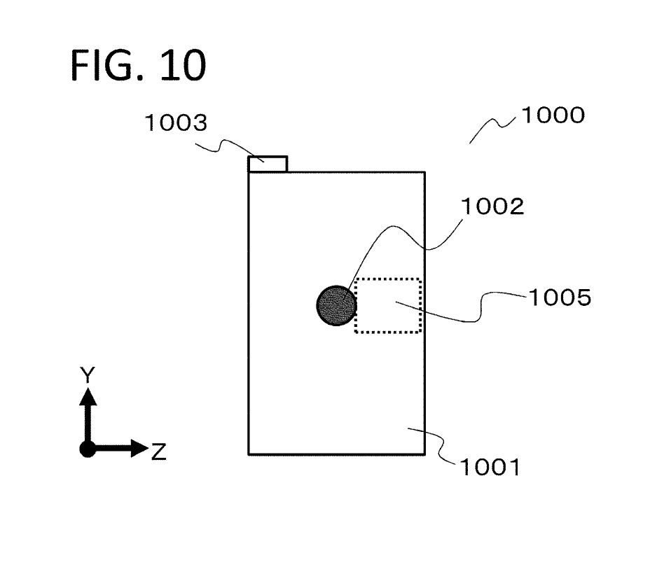

FIG. 10 is a phantom 1000 according to the present embodiment. In a conventional phantom, when reconstructing an ultrasound image, an image is generated assuming that the speed of sound is uniform between manufacturing lots of the phantom and the speed of sound distribution within the phantom is uniform. In other words, when a plurality of phantoms are manufactured under the same conditions, it is presumed that, in an initial state, the same acoustic property values (here, sound speed values) apply. However, according to research by the present inventors and others, fluctuation of approximately 30-40 m/s occurs in the speed of sound upon shipment, between lots of the phantoms, depending on the water content. Moreover, even within the same phantom, variations in the sound speed value of approximately 2-3 m/s occur depending on the position, due to non-uniformities in the compound having light scattering properties, which is typically titanium oxide. There is a risk that these fluctuations in the speed of sound may cause decline in the spatial resolution and deterioration in the image quality due to distortion.

Therefore, in the phantom 1000 according to the present embodiment, provided inside a base material 1001 are: an absorber 1002; and an identification tag 1003 which stores information about the acoustic property values of at least a portion of the base material 1001. The stored acoustic property values are acoustic property values, such as the speed of sound, attenuation, acoustic impedance, and the like, in the phantom. These values may be set for each phantom individually, or for respective positions within one individual phantom. The initial acoustic property values upon shipment of the phantom and the related positional information (for example, the coordinates inside the phantom) are stored in associated fashion, in the identification tag. Alternatively, initial acoustic property values for each individual phantom are stored. The location 1005 inside the phantom indicates a portion of the phantom where the acoustic property values are measured. The location 1005 inside the phantom may be a location which includes the absorber, and not only the base material.

The identification tag may store average acoustic property values for the whole of the phantom, or for the whole portion of the base material. Furthermore, the portion may be one point within the phantom. The portion may also be a region having a volume, a thin sheet-shaped region, or a line-shaped region. In this case, the average acoustic property value in the region may be used as the acoustic property value. It is especially desirable if the portion is a region on the path from the absorber to the probe, which has a large effect on image quality.

The information processing apparatus according to the present embodiment sets the acoustic property values to be used for calibration, on the basis of the information in the identification tag. For example, when a sound speed value, and positional information for the location in the phantom corresponding to that sound speed value, has been read in, the corresponding numerical values are determined when image reconstruction is carried out by processing the signal from the absorber. For example, these values can be used as coefficients in the signal intensity or coefficients in the obtained image. The positional information is two-dimensional or three-dimensional information, and is set in accordance with a distance (cm or mm, etc.), or a number of voxels or sign thereof. Consequently, since the arrival time of the photoacoustic wave is acquired on the basis of the actual sound speed value, and an image is reconstructed, then the accuracy of the image is improved.

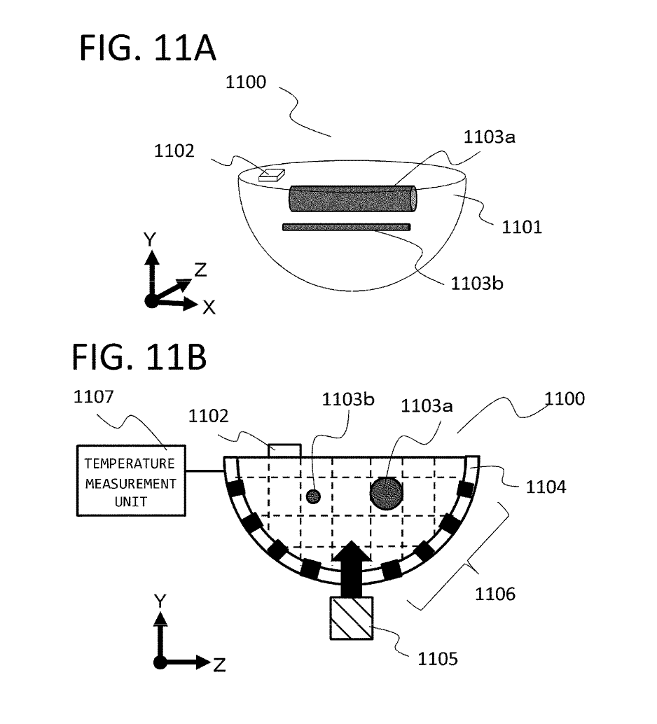

FIG. 11A is an example of a phantom 1100 according to the present embodiment. Arranged inside the phantom are a base material 1101, absorbers 1103a, 1103b, and an identification tag 1102. The absorbers are two rod-shaped absorbers, which have cylindrical shapes of different thicknesses.

FIG. 11B is a cross-sectional diagram of a phantom. The whole of this phantom is divided in sections of a cuboid shape having edges of 5 mm, and the acoustic property values in each of the sections are stored in the identification tag 1102 in association with both positional information and the phantom temperature. A hemispherical phantom 1100 is held in a spherical surface type holding section 1104. A light source 1105 irradiates light vertically upwards. An acoustic wave probe 1106 is arranged along the spherical surface type holding section.

A temperature measurement unit 1107 is, for instance, a radiation thermometer for thermography, which measures the temperature of the phantom for use in setting the acoustic property values for image reconstruction. The information processing apparatus acquires the acoustic property values corresponding to the temperature and positional information, by referring to a table, or the like, and uses this information for image reconstruction. In this way, according to the present embodiment, the non-uniformity in a portion of the phantom, which is a cause of decline in the accuracy of reconstruction, can be corrected by the information in the identification tag.

### Seventh Embodiment

With a conventional phantom, there are cases where the optical property values are non-uniform in the manufacturing process. In other words, when a plurality of phantoms are manufactured under the same conditions, fluctuation occurs between manufacturing lots, and there is error in the initial optical property values. Error in the optical property values which occurs depending on the location within the phantom may cause a decline in the accuracy of the phantom image during image reconstruction.

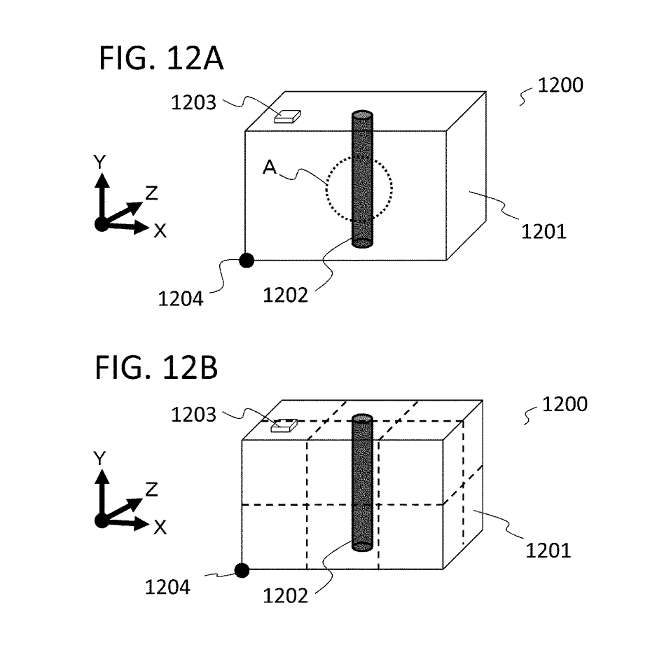

Therefore, the phantom 1200 according to the present embodiment which is shown in FIG. 12A is provided with an absorber 1202 arranged inside a base material 1201, and an identification tag 1203 which stores information about the optical property values of at least the partial region (A) of the base material 1202. This identification tag contains, as initial information, the optical property values of at least one location in the phantom, and the positional information of the location, in associated fashion. The optical property values referred to here are, for example, the absorption coefficient, the reduced scattering coefficient, the refractive index, the reflectivity or the transmissivity, etc. One example of a method for recording the positional information is a method which specifies a location by a distance and angle from a reference point, which is indicated by reference numeral 1204.

The identification tag may store average optical property values for the whole of the phantom, or for the whole portion of the base material. Furthermore, the portion may be one point within the phantom. The portion may also be a region having a volume, a thin sheet-shaped region, or a line-shaped region. In this case, the average optical property value in the region may be used as the optical property value. It is especially desirable if the portion is a region on the path from the optical member for light irradiation to the absorber, which has a large effect on image quality.

For the method of acquiring optical property values, it is possible to employ Time-ReSolved SpectroScopy (TRS), or intensity modulation, or a spectrophotometer. The acquired optical property values are stored as initial information in the identification tag. Optical property values may be acquired from a plurality of regions within the phantom. For example, in FIG. 12B, optical property values are acquired by dividing the phantom into 6 regions.

The items which are calibrated by using the optical property values associated with the positional information may be the quantity of light irradiated from the light source, the angle of light irradiation onto the object, the transparency or positional accuracy of the object holding section. Moreover, the calibrated items may also be the detection sensitivity, position or angle of the probe, the speed or positional accuracy of the scanner, or the amplification rate of the signal processing unit, and so on.

When performing image reconstruction, the absorption coefficient and the reduced scattering coefficient, which are optical property values, affect the light intensity and light quantity distribution of the light transmitted inside the object, after being irradiated from the light source. By accurately estimating the quantity of light in a target voxel, by using the identification tag information according to the present embodiment, the accuracy of calculation of the absorption coefficient based on the initial sound pressure is improved.

### Eighth Embodiment

The phantom according to the present embodiment is provided with a two-dimensional code which stores initial information upon shipment. The initial information upon shipment is information that is unique to the phantom, for example, the date of manufacture of the phantom, the site of manufacture, the manufacture lot number, and the like. The initial information also includes the optical property values, the acoustic property values, mode information, surface profile information, contained tangible object information, defect-related information of the phantom, and the like.

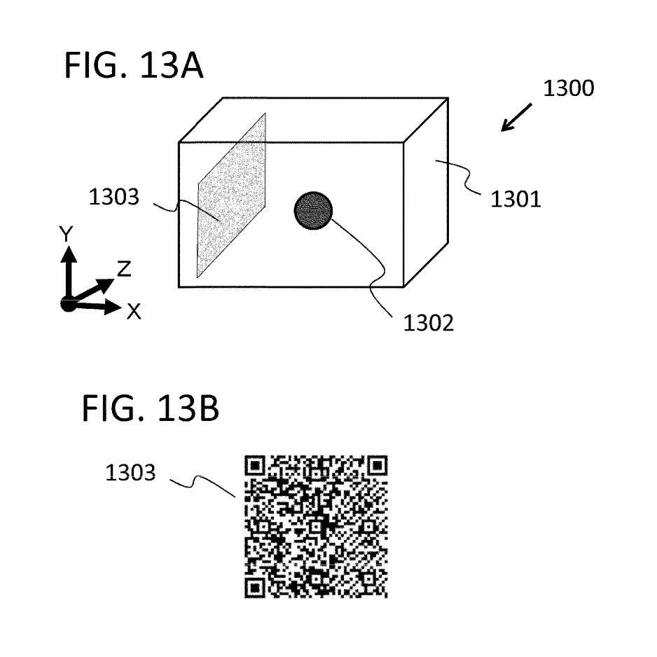

FIG. 13A is a diagram illustrating the configuration of a phantom 1300 according to the present embodiment. The phantom 1300 has a light absorber 1302 and a tag section 1303 included inside a base material 1301. FIG. 13B is a diagram for describing the tag section 1303. The tag section is a two-dimensional code in which a pattern is formed by a material having a strong absorptivity in respect of the wavelength of the measurement light that is irradiated from the photoacoustic measurement apparatus. The two-dimensional code in FIG. 13B is a QR code (registered trademark). More specifically, the portion represented by a dark color (such as black) in the QR code is formed from a material having a different light absorptivity to the phantom base material. A material of this kind may be, for example, a pigment, a dye, carbon black material, or the like. The two-dimensional code maybe formed, for example, by burying line-shaped structures that absorb light at a prescribed width and interval apart, or may be formed by printing with an inkjet printer onto a transparent medium, such as an OHP sheet. Provided that the material has a strong absorptivity in respect of the wavelength of the irradiated light, then any material may be used and the color thereof does not have to be black.

The bit size of the two-dimensional code of the tag section should be set in accordance with the resolution of the photoacoustic measurement apparatus used. For example, if the apparatus has a resolution of 0.5 mm, then in order to read in information accurately, the bit size is desirably set to no less than 0.5 mm. Furthermore, the same applies to the thickness of the two-dimensional code.

For example, if the two-dimensional code employed has 37 modules on one edge and a bit size of 1 mm, then in the case of the present embodiment, the code will be a 37-mm square. In other words, the code will be of a size that can be embedded in the phantom. Here, if the error correction level is set to H (maximum), then it is possible to hold a maximum of 106 text characters by taking the abovementioned information to be a list of numbers.

In the information described above, by setting the physical values of the phantom base material and the physical values of the absorber as calibration information, and introducing “0000” as a value between each of the items, as indicated below, then a 96-character number list such as the following is obtained. “370000137900001.4200000.52000079700000.00400000.93000037000 01.6300001.08000079700000.09800000.92”

FIG. 13B shows this list in coded form.

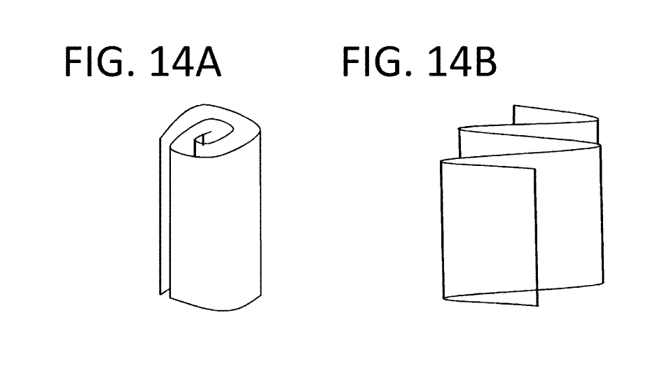

Furthermore, it is also possible to use a two-dimensional code formed on a curved surface, rather than a flat planar two-dimensional code, as the tag. For example, FIG. 14A is a two-dimensional code formed in a roll shape and FIG. 14B is a two-dimensional code formed in an undulating shape. Furthermore, if the object holding section has a spherical surface profile, then the two-dimensional code may be arranged in a curved shape, following the spherical surface. Since the photoacoustic measurement apparatus is capable of creating an image of three-dimensional information in the object, the apparatus is able to read in information arranged at any depth. Furthermore, the information read in can be developed and decoded on the system side. In other words, it is possible to impart a large amount of information to a region of a limited size. Furthermore, it is also possible to layer a plurality of two-dimensional codes. Consequently, the surface area per code can be reduced and the region inside the phantom can be utilized efficiently. Furthermore, one-dimensional bar codes, text characters and symbols can be arranged directly in the phantom. Moreover, a code other than a QR code (for example, code encoded by any other coding algorithm) maybe used as the two-dimensional code. Furthermore, two-dimensional codes may be layered three-dimensionally, and one-dimensional codes may also be layered three-dimensionally.

According to the present embodiment, an identification tag containing information required for measurement can be generated as a portion of a phantom. Since the image calibration function which is generally provided in the image can be used for reading the identification tag information, then it is possible to reduce costs. The method according to the present embodiment can be applied to several identification tags of the respective embodiments described above.

### Ninth Embodiment

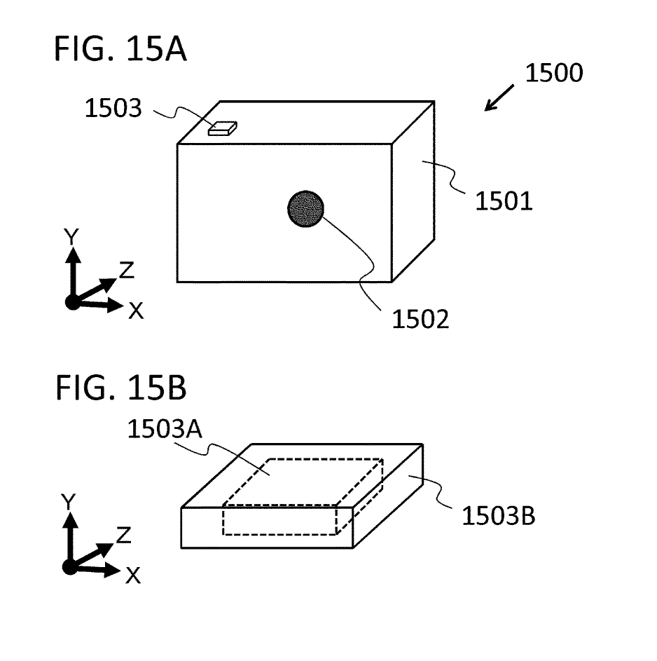

FIG. 15A is a diagram illustrating the configuration of a phantom 1500 according to the present embodiment. In the phantom, a light absorber 1501 is arranged inside a phantom base material 1501 which simulates object tissue, and an identification tag 1503 is arranged on the upper surface thereof.

Desirably, the identification tag 1503A is covered with a material having high reflectivity in respect of the wavelength of the measurement light which is irradiated from the photoacoustic measurement apparatus. For the material of this kind, it is possible to use, for example, a metal such as silver, copper or aluminum, or a dielectric multi-layer film. The materials stated above have a reflectivity of 80% or above with respect to measurement light having a wavelength in the near-infrared region. In the present embodiment, the whole surface of the identification tag 1503A is covered by using a reflector 1503B made from the materials described above. The identification tag 1503A may be covered directly by the reflector 1503B, or may be covered by the reflector 1503B after forming a protective layer on the surface of the identification tag 1503A.

The identification tag 1503A itself may also be made from a material having high reflectivity. Furthermore, the reflector 1503B does not have to cover the entire surface of the identification tag, provided that the reflector is capable of shielding measurement light irradiated from outside and suppressing the occurrence of unwanted acoustic waves. Silver, copper, aluminum, and the like, are given as examples of a material for the reflector 1503B, but other materials maybe used provided that the member has a reflectivity equal to or greater than a prescribed value in respect of light irradiated from outside (a reflectivity capable of limiting the occurrence of acoustic waves to a certain level or less). It is particularly suitable to use a member having high reflectivity in respect of near-infrared light which is used by the photoacoustic measurement apparatus.

By adopting a configuration of this kind, acoustic waves generated from the identification tag itself are suppressed, and a favorable image can be obtained. The reflector according to the present embodiment can be used in each of the identification tags holding various information which were described in the respective embodiments above.

### Modification Examples

The descriptions of the respective embodiments are examples for the purpose of explaining the present invention, and the present invention can be implemented by appropriately modifying or combining these embodiments, without departing from the essence of the invention. The present invention can also be implemented as an object information acquiring system provided with an object information acquiring apparatus and the phantom for an object information acquiring apparatus described above. The abovementioned processing and units can be implemented by combining same freely, provided that no technical contradictions arise. For example, the reduction in the photoacoustic waves from the identification tag and the identification of the positions of artifacts using positional information for tangible objects in the identification tag may be carried out simultaneously, or may be carried out individually.

### Other Embodiments

Embodiment (s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2014-124376, filed on Jun. 17, 2014, Japanese Patent Application No. 2014-124377, filed on Jun. 17, 2014, Japanese Patent Application No. 2014-124378, filed on Jun. 17, 2014, Japanese Patent Application No. 2014-124379, filed on Jun. 17, 2014, Japanese Patent Application No. 2014-124380, filed on Jun. 17, 2014, and, Japanese Patent Application No. 2014-124381, filed on Jun. 17, 2014, which are hereby incorporated by reference herein in their entirety.

1. An object information acquiring apparatus which uses a phantom having a base material, an absorber arranged inside the base material, and an identification tag, the apparatus comprising:
a probe which receives an acoustic wave and outputs an electrical signal;
a reader which reads in information from the identification tag;
a generator which generates information for performing calibration, by using an electrical signal output by the probe as a result of receiving an acoustic wave propagated from the phantom, and information read out from the identification tag by the reader; and
a processor which acquires property information of the inside of an object by using the electrical signal output by the probe as a result of receiving the acoustic wave propagated from the phantom,
wherein the identification tag stores information relating to the phantom, the information being used by the processor when acquiring the property information.

2. The object information acquiring apparatus according to claim 1, further comprising a calibrator which performs calibration of the object information acquiring apparatus by using information generated by the generator.

3. The object information acquiring apparatus according to claim 1, wherein the identification tag stores information about at least one of a position, a size and a shape of a tangible object which is contained in the base material and is other than the absorber.

4. The object information acquiring apparatus according to claim 1, wherein the identification tag stores information relating to a surface profile of the phantom.

5. The object information acquiring apparatus according to claim 4, wherein the identification tag stores information about at least one of partial omissions in the base material, defects in the base material, and unevenness in the base material.

6. The object information acquiring apparatus according to claim 1, wherein the identification tag stores information relating to acoustic property values of at least a portion of the phantom, in association with positional information for the portion.

7. The object information acquiring apparatus according to claim 1, wherein the identification tag stores information relating to optical property values of at least a portion of the phantom, in association with positional information for the portion.

8. The object information acquiring apparatus according to claim 1, further comprising a display which displays a phantom image indicating the property information of the phantom acquired by the processor.

9. The object information acquiring apparatus according to claim 1, further comprising a light source which generates the acoustic wave by irradiating light onto the object, wherein the generator generates information for controlling at least one of a quantity of light, an irradiation angle and an irradiation position of the light source, as the information for performing the calibration.

10. The object information acquiring apparatus according to claim 1, further comprising a scanner which performs a scanning action of the probe,
wherein the generator generates information for controlling a speed of scanning by the scanner, as the information for performing the calibration.

11. The object information acquiring apparatus according to claim 1, wherein the generator generates information for controlling at least one of a detection sensitivity, a position and an angle of the probe, as the information for performing the calibration.

12. The object information acquiring apparatus according to claim 9, wherein the identification tag stores the information by three-dimensionally arranging a prescribed pattern having differences in light absorptivity.

13. The object information acquiring apparatus according to claim 9, wherein the identification tag has a reflector which suppresses the generation of acoustic waves by reflecting light irradiated from outside.

14. A phantom used for calibration of an object information acquiring apparatus which acquires property information of the inside of an object on the basis of an acoustic wave propagated from the object, the phantom comprising:
a base material;
an absorber arranged inside the base material; and
an identification tag which stores information relating to the phantom, the information being used by the object information acquiring apparatus when acquiring the property information.

15. A method for manufacturing a phantom used for calibration of an object information acquiring apparatus which acquires property information of the inside of an object on the basis of an acoustic wave propagated from the object, the method comprising steps of:
forming a base material;
arranging an absorber inside the base material;
arranging an identification tag in the base material; and
storing information relating to the phantom in the identification tag, the information being used by the object information acquiring apparatus when acquiring the property information.
